(19) United States
(12) Patent Application Publication
(10) Pub. No.: US 2007/0282178 A1
(43) Pub. Date: Dec. 6, 2007

(54) Method and device for the identification of at least one substance of content of a body fluid

(75) Inventors: Bernd Scholler, Karlsruhe (DE); Karl-Andreas Feldhahn, Hamburg (DE); Klaus Forstner, Asperg (DE)

Correspondence Address: Friedrich Kueffner, Suite 910, 317 Madison Avenue, New York, NY, 10017, US

(73) Assignee: Weinmann Gerate fur Medizin GmbH & Co. KG

(21) Appl. No.: 11/787,105

(22) Filed: Apr. 12, 2007

(30) Foreign Application Priority Data
Apr. 12, 2006 (DE) 10 2006 018 044.5

(51) Int. Cl. A61B 5/1455

(52) U.S. Cl. 600/310

(57) ABSTRACT

A method and a device for the identification of at least one substance of content of a body fluid, wherein adjacent to a body tissue containing the body fluid at least one radiation source and a photo receiver are arranged. The radiation source generates radiation of at least two different wavelengths and the radiation is directed onto the body tissue. The photo receiver receives radiation reflected by the body tissue and/or reduced through the body weight. At least at one point radiation from two radiation sources with an essentially same wavelength is directed essentially simultaneously onto the body tissue for penetration and/or reflection.

### BACKGROUND OF THE INVENTION

1. Field of the Invention

The present invention relates to a method and a device for the identification of at least one substance of content of a body fluid, wherein adjacent to a body tissue containing the body fluid at least one radiation source as well as a photo receiver are arranged, wherein the radiation source generates radiation of at least two different wavelengths, wherein the radiation is directed onto the body tissue and the photo receiver receives radiation that is reflected by the body tissue and/or is reduced through the body tissue.

2. Description of the Related Art

It is known in the art to conduct a radiography and/or a backscatter at a desired wavelength with a predeterminable intensity on body tissue with wavelength-dependent absorption coefficients and/or with strong light dispersion, in which an adjustment desired and/or predetermined by the user can be conducted manually or through automatic regulation.

The long established pulsoximetry allows for a non-invasive measurement of the oxygen saturation of the arterial blood. For this, for example, the light of two different wavelengths, for example, 660 nm and 905 nm is guided through a finder, and which is partially absorbed by the blood pulsating through the tissue. The degree of absorption is defined through an analysis of the portion of the light exiting on the other side of the radiographed tissue, which allows an immediate conclusion as to the oxygen saturation of the pulsating and thus arterial blood.

The pulse spectroscopy expands the non-invasive diagnostic, among other things, by the following blood parameters: concentration of hemoglobin, absolute oxygen saturation of the blood, carbon monoxide concentration, concentration of methemoglobin, concentration of bile pigment. When conducting a pulse spectroscopy, like in a pulsoximetry, also wavelengths of, for example, 660 nm and 905 nm are used, however, further wavelengths are necessary. The principles of the pulse spectroscopy are illustrated in the following patent documents: DE 103 21 338 A1, DE 102 13 692 A1 and DE 10 2005 020 022 A1.

In media with wavelength-dependent absorption the intensity of the radiation changes with the distance and the spectral composition. This is also true for the scattering of the radiation, because it weakens the radiation due to the size and the number of the dispersion centers and it also changes the radiation spectrally with distance. Therefore, radiation sources are needed that can optimally compensate these changes in order to facilitate an evaluation of the reflected back dispersed portion and/or of the portion after a radiography.

Such changes of radiation are caused, for example, by a wavelength dependent absorption of the substances of content of a body fluid like, for example, hemoglobin, glucose, bile pigment, and water which can be described by approximation through the Beer Lambert law.

The absorption of radiation of a defined wavelength can be quickly estimated with the help of the absorption coefficient. The absorption coefficient of water shows strong wavelength dependency. Water molecules show a strong absorption band at approximately 1450 nm.

Hemoglobin, for example, has two transmission bands in the red and in the blue-green zone.

### SUMMARY OF THE INVENTION

It is the object of the present invention to create a method and a device which during the identification of substances of content of a body tissue automatically provides the degree of intensity of the radiation source dependent on the absorption and/or reflection, and which changes the radiation characteristics according to necessity and also facilitates a minimized requirement of energy.

The source of electromagnetic radiation is, for example, one or several laser diodes and/or one or several white light sources and/or one or several LED.

The object of the invention is solved by using different light emitting diodes (LED) with same and/or different configuration. The use of light emitting diodes guarantees, on the one hand, a long life span and a low power consumption so that at least two of the above-mentioned conditions would already be fulfilled. The invention is characterized by additional features that take as much advantage of the good activation characteristics as well as its emission characteristics and its different radiation characteristics.

A solution is provided with which a non-invasive identification of at least one substance of content of a body fluid chosen from the group of pulse frequency, ph-value, concentration of hemoglobin (cHb), oxyhemoglobin (HbO2), desoxygenized hemoglobin (HbDe), carboxyhemoglobin (HbCO), methemoglobin (cMetHb), sulfhemoglobin (HbSulf), bile pigment, glucose, bile pigments, SaO2, SaCO, SpO2, CaO2, SpCO, is made possible. Furthermore, a non-invasive identification of several substances of content of a body fluid is possible.

An important feature of light emitting diodes for the realization is their activation through their non-linear power-voltage characteristic curve according to the Shockley equation.
I=Iexp (Uf/nkT)

I: flow stream; UF: flow tension; I: saturation flow; k: Boltzmann constant; T: absolute temperature, n: constant (with a value between 1 and 2).

Since the number of emitted photons over a great flow area is directly proportional to the flow stream, LED are easily controlled over several ranges concerning their light intensity through a small change in the flow tension.

Theoretically, changes in the flow tension of up to 150 mV are possible. This would cause a change of flow tension by factor 10 and a change of luminosity also by 10.

GaAIAs/GaAs (red and infrared): 1.2 to 1.8 V

InGaAIP (red and orange): 2.2 V

GaAsP/GaP (yellow): 2.1 V

GaP/GaP (green): 2.1 V

InGaN (blue and white): 3.3 to 4 V

Silicon diode: 0.7 V

The power input varies from one model to another between 2 mA, 20 mA (for example 5-mm-LED) up to approximately 700 mA or more in LED for purposes of illumination. The conducting state voltage (Uf) hereby ranges from approximately 1.5 V (infrared-LED) to approximately 4 V (InGaN-LED: green, blue UV).

This creates the possibility, when using different LED, to quickly manage and purposefully change an additive complement of the luminosity/light intensity by targeted regulation of one kind of LED.

Thus it is possible, in selective absorption as it can occur in water or in blood (through hemoglobin), to control one kind of LED current-wise by using different LED in such a manner that different tissue thicknesses, skin pigmentations and other factors are considered in such a way that a photo receiver always receives a defined portion of scattered radiation and/or reduced radiation for evaluation.

A further characteristic of LED is its varied irradiation characteristic which can show aperture angles from 20° to 45°; in addition, almost cosine-like irradiation is possible.

The various features of novelty which characterize the invention are pointed out with particularity in the claims annexed to and forming a part of the disclosure. For a better understanding of the invention, its operating advantages, specific objects attained by its use, reference should be had to the drawing and descriptive matter in which there are illustrated and described preferred embodiments of the invention.

### BRIEF DESCRIPTION OF THE DRAWING

In the drawing:

FIG. 1 is a schematic illustration of an LED arrangement;

FIG. 2 is a further schematic illustration of an LED arrangement;

FIG. 3 is schematic illustration of a finger clip sensor;

FIG. 4 shows a typical absorption process in a measuring of blood and water;

FIG. 5 shows absorption spectrums of functional and dysfunctional hemoglobin derivates; and

FIG. 6 shows a typical process of the absorbancy coefficient for various hemoglobin derivates.

### DETAILED DESCRIPTION OF THE INVENTION

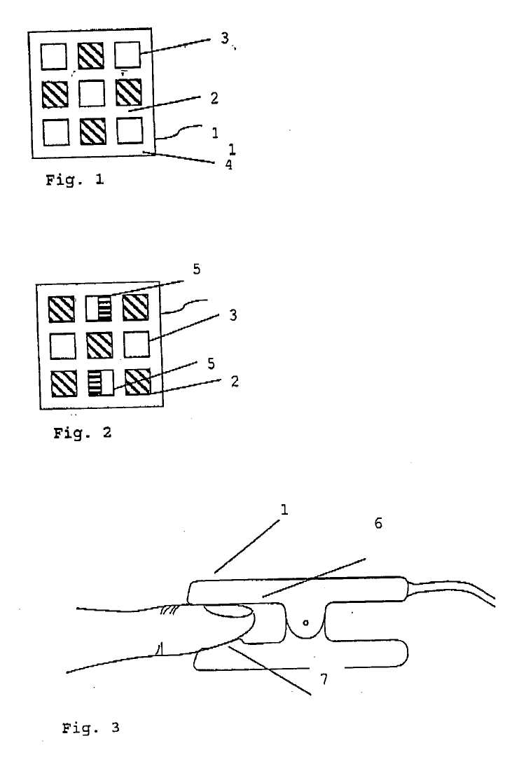

The LED arrangement 1 as shown in FIG. 1 and FIG. 2, respectively, includes numerous LED which are mounted on a collective carrier 4, for example, a circuit board with adequate conduct structures (not shown) for the electrical supply and the activation of the LED. Alternatively, the carrier can also be designed as a finger clip sensor.

FIG. 2, in addition, shows an LED 5 which emits two wavelengths.

FIG. 3 shows a finger clip sensor 6 with integrated LED arrangement 1 and photo receiver 7.

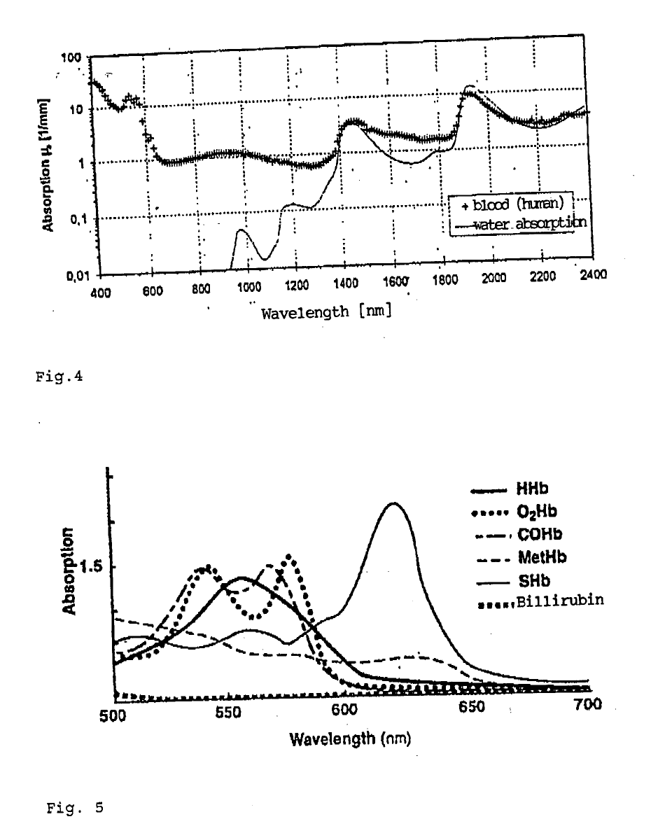

FIG. 4 shows a typical absorption process for the measuring of blood and water. One recognizes absorption maxima for water in the range of wavelengths of 950 nm, 1200 nm, 1450 nm, 1900 nm and 2400 nm. One recognizes absorption maxima for blood in the range of wavelengths of 550 nm, 910 nm, 1450 nm and 1900 nm.

FIG. 5 shows a typical absorption process for the measuring of oxygen saturation in blood. An absorption intensity is applied in dependence on the respective wavelength. A first minimum is encountered at a wavelength of approximately 600 nanometer. Starting at approximately 680 nanometer, the progression approaches asymptotically the zero line.

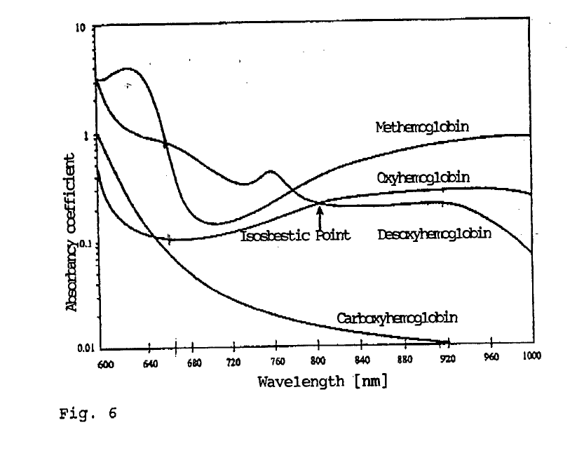

FIG. 6 shows a typical process of the typical course of the absorbancy coefficients for various hemoglobin derivates. At 805 nm is the isobestic point, here the absorbancy of oxyhemoglobin is equal to the absorbancy of desoxyhemoglobin.

The LED are respectively connectable with an LED control device. The LED control device regulates the power and/or voltage supply of each individual LED.

The LED are covered with a coating (not shown).

The LED have at least two different emission wavelengths. According to the invention, there are at least two LED for every emission wavelength in the area of the LED device. One of the two LED for one emission wavelength is the main LED, the at least one further LED of the same emission wavelength serves as auxiliary LED.

By means of these auxiliary light emitting diodes 3 those spectral components are added to the over-all spectrum that are missing in the emission spectrum of the active main LED 4 and/or that are not available at a sufficient strength.

Preferably, the main and/or auxiliary LED are configured in such a way that they can emit alternatively and/or complementary the following wavelengths selected from the group:

150 nm±15%, 400 nm±15%, 460 nm±15%, 480 nm±15%, 520 nm±15%, 550 nm±15%, 560 nm±15%, 606 nm±15%, 617 nm±15%, 620±15%, 630 nm±15%, 650 nm±15%, 660 nm±15%, 705 nm±15%, 710 nm±15%, 720 nm±10%, 805 nm±15%, 810 nm±15%, 880 nm±15%, 890 nm, 905 nm±15%, 910 nm±15%, 950 nm±15%, 980 nm±15%, 980 nm±15%, 1000 nm±15%, 1030 nm±15%, 1050 nm±15%, 1100 nm±15%, 1200 nm±15%, 1310 nm±15%, 1380 nm±15%, 1450 nm±15%, 1600 nm±15%, 1650 nm±15%, 1670 nm±15%, 1730 nm±15%, 1800 nm±15%, 2100 nm±15%, 2250 nm±15%, 2500 nm±15%, 2800 nm±15%

TABLE 1

| Wavelength | |
|---|---|
| (nm) | LED material |
| 940 | GaAIAs/GaAs |
| 880 | GaAIAs/GaAs |
| 850 | GaAIAs/GaAs |
| 660 | GaAIAS/GaAs |
| 635 | GaAsP/GaP |
| 633 | InGaAIP |
| 620 | InGaAIP |
| 612 | InGaAIP |
| 605 | GaAsP/GaP |
| 595 | InGaAIP |
| 592 | InGaAIP |
| 585 | GaAsP/GaP |
| 574 | InGaAIP |
| 570 | InGaAIP |
| 565 | GaP/GaP |
| 560 | InGaAIP |
| 555 | GaP/GaP |
| 525 | SiC/GaN |
| 505 | SiC/GaN |
| 470 | SiC/GaN |
| 430 | SiC/GaN |
| 660/910 | AIGaAs |
| 660/850 | |
| 660/940 | |
| 635/760 | |
| 565/660 | |
| 760/940 | |

Table 1 shows an exemplified list of suitable light emitting diodes, that can be used in accordance with the invention.

According to the invention, al least two LED of a wavelength range are used in an LED configuration. This redundancy regarding the wavelength range makes it possible to compensate for the breakdown of singular LED and/or to chose an alternative radiation entry area for a wavelength range and/or to increase the intensity for one wavelength range through simultaneous use of at least two LED.

According to the invention, it has also been considered to use two-wavelengths emitting LED. According to the invention it is preferred to use such two-wavelengths emitting LED in which the intensity of each of the two wavelengths can be controlled independently.

For example, the main LED emits in the area of, for example, 1450 nm±15%. Due to a thick tissue layer of the examined finger, the leftover intensity of the radiation after passing through the tissue is no longer sufficient for an evaluation. To begin with, the radiation intensity (at 1450 nm) of the main LED can be increased manually and/or automatically through the LED control device. Alternatively and/or complementary an auxiliary LED, that also emits in the area of 1450 nm, can be additionally connected. It is also provided that the auxiliary LED emits at a wavelength range±15% of the wavelength of the main LED. According to the invention, the auxiliary LED is preferably arranged in the area of the LED configuration at a distance of at least 1 mm from the main LED. Through the additional connection of the auxiliary LED, the leftover intensity after passing through the tissue is again sufficient for evaluation.

In another embodiment, the main LED emits in the range of, for example, 660 nm±15%. Due to a local intensive pigmentation in the radiation area of the examined finger, the leftover intensity of the radiation after passing through the tissue is no longer sufficient for evaluation.

First of all, the radiation intensity (at 660 nm) of the main LED can be increased manually or automatically via the LED control device. Alternatively and/or additionally, an additional LED, that also emits in the realm of 660 nm±15%, can be connected. Because the auxiliary LED is arranged at a distance of at least 1 mm from the main LED, the auxiliary LED radiates outside of the local intensive pigmentation. The radiation of the auxiliary LED passes through the finger at a sufficient leftover intensity and an evaluation is possible.

In another embodiment, the main LED emits in the range of infrared 890 nm±15% or 910 nm±15%. Due to a defect in the main LED, the LED control device activates an auxiliary LED which also emits in the infrared realm. Because of the redundance of light emitting diodes that emit in the range of a wavelength, a failing LED can be compensated by another LED of the same wavelength.

While specific embodiments of the invention have been shown and described in detail to illustrate the inventive principles, it will be understood that the invention may be embodied otherwise without departing from such principles.

We claim:

1. A method for the identification of a substance of content of a body fluid, the method comprising arranging adjacent to a body tissue containing the body fluid at least one radiation source and a photo receiver, wherein the radiation source generates radiation of at least two different wavelengths, directing the radiation onto the body tissue the photo receiver receiving radiation that is reflected by the body tissue and/or receiving radiation that is reduced through the body tissue, directing at least at one point in time radiation of essentially the same wavelength from at least two radiation sources essentially synchronously onto the body tissue for penetration and/or reflection.

2. The method according to claim 1, comprising, upon the failure of a main radiation source, compensating the wavelength of the main radiation source by means of an auxiliary radiation source.

3. The method according to claim 1, comprising, upon the failure of a main radiation source, compensating an intensity of the main radiation source by means of an auxiliary radiation source.

4. The method according to claim 1, comprising arranging the main radiation source and the auxiliary radiation source at a distance of at least 1 mm from each other.

5. The method according to claim 1, wherein at least two radiation sources emit at an essentially identical wavelength, and wherein, by choosing the emitting radiation source, an alternative radiation focus point for the wavelength range can be chosen.

6. The method according to claim 1, wherein at least two radiation sources emit at an essentially same wavelength range, and wherein the intensity for one wavelength range can be enhanced by using both radiation sources simultaneously.

7. A device for the identification of at least one substance of content of a body fluid, the device comprising at least one radiation source for the generation of radiation of different wavelengths, and a photo receiver, wherein the radiation source and the photo receiver are connected to a clamping device for positioning within the area of a body tissue containing the body fluid, wherein at least two radiation sources are configured to emit at least at one point at essentially equal wavelengths at the body tissue.

8. The device according to claim 7, wherein the radiation sources are configured as radiation emitting diodes (LED).

9. The device according to clam 7, wherein a majority of LED with different emission wavelengths are arranged in a common housing.

10. The device according to claim 7, wherein the light emitting diodes can be individually activated.

11. The device according to claim 7, wherein at least two LED emit in the red spectrum zone.

12. The device according to claim 7, wherein at least two LED emit in the infrared spectrum zone.

13. The device according to claim 7, wherein at least two LED emit in the realm of a wavelength in which a high absorption of water is present.

14. The device according to claim 7, wherein one principal LED emits with a predetermined spectrum, and at least one auxiliary LED completes certain spectrum portions temporarily.

15. The device according to claim 7, wherein a principal LED emits radiation with a predetermined spectrum, wherein at least one auxiliary LED complements certain intensities in the realm of the spectrum of the principal LED.

16. The device according to claim 7, wherein via power regulation of an LED selectively absorbing and/or scattering media can be illuminated with a predetermined intensity, wherein the power regulation activates at least one further LED for increase of intensity according to necessity.

17. The device according to claim 7, wherein at least two LED with different coloration with a small opening angle are chosen which can be controlled via electrical power in such a way that, after passing a selectively absorbing and/or scattering media, leftover intensity is present at a photo receiver at a definable distance which is sufficient for evaluation purposes.

18. The device according to claim 7, wherein through a small deviation in the flow tension the intensity of the light of the radiation source can be changed by more than two orders of magnitudes and the scope can be controlled within a wide range according to the necessities of the user.

19. The device according to claim 7, wherein, depending on the respective effective selective absorption coefficients, also a selective activation of the present light emitting diodes is conducted for the attainability of a sufficiently high leftover intensity at a photo receiver.

20. The device according to claim 7, wherein, in order to attain a sufficiently high leftover intensity at a photo receiver, there are only used LED with a spectrum that is within the absorption minimum and/or the absorption maximum of the present medium.

21. The device according to claim 7, wherein at low leftover intensity at a photo receiver successively light emitting diodes with an emission that lies further in the long wave spectrum are drawn on.

22. The device according to claim 7, wherein at low leftover intensity at a photo receiver successively light emitting diodes with an emission that lies further in the short wave spectrum are used.

23. The device according to claim 7, comprising light emitting diodes emitting two wavelengths.

24. A device for the identification of at least one substance of content of a body fluid, the device comprising at least two radiation sources for the generation of radiation of different wavelengths and a photo receiver, wherein at least at one point in time radiation of essentially equal wavelengths in the range of 1450±10% is directed essentially simultaneously onto the body tissue.

25. A device for the identification of at least one substance of content of a body fluid, the device comprising at least two radiation sources for the generation of radiation of different wavelengths and a photo receiver, wherein at least at one point in time radiation of essentially equal wavelengths in the range of 660±10% is directed essentially simultaneously onto the body tissue.

26. The device according to claim 7, wherein a principal LED emits radiation with a given spectrum, and wherein this spectrum has a first high absorption for a blood parameter and/or water, and wherein at least one auxiliary LED emits at a certain wavelength at least temporarily and/or simultaneously, and wherein this wavelength has a second high absorption for a blood parameter and/or water.

27. The method according to claim 1, wherein a predetermined spectrum is emitted from a principal LED and wherein this spectrum is predetermined in such a way that the spectrum has a first high absorption for a blood parameter and/or for water, and wherein at least from one auxiliary LED a certain wavelength is emitted at least temporarily and/or simultaneously with the emission from the principal LED, and wherein at this wavelength a second high absorption for a blood parameter and/or water is present.
